(19) United States
(12) Patent Application Publication
(10) Pub. No.: US 2014/0046270 A1
(43) Pub. Date: Feb. 13, 2014

(54) PREFILLED MEDICAL INJECTION DEVICE

(71) Applicant: Daniel W. Thornton, Denver, CO (US)

(72) Inventor: Daniel W. Thornton, Denver, CO (US)

(73) Assignee: ADAR MEDTECH, INC., Marina Del Rey, CA (US)

(21) Appl. No.: 13/870,815

(22) Filed: Apr. 25, 2013

Related U.S. Application Data
(60) Provisional application No. 61/638,059, filed on Apr. 25, 2012.

(51) Int. Cl. A61M 5/24; A61M 5/168

(52) U.S. Cl. CPC A61M 5/2455; A61M 5/168
USPC 604/244; 604/256

(57) ABSTRACT

An integrated injection device with pre-packaged medication is disclosed. The integrated injection device includes an ampule that is filled with a fluid medication and a collar with hypodermic needle. The collar includes an axial valve that is positioned between a first end of the collar a second end that includes an exit aperture that is connected to the hypodermic needle. The valve is movable from a first position where the valve blocks fluid communication between the first end and the second end and a second position where valve provides fluid communication between the first end and the exit aperture in the second end. The valve cooperates with a frangible webbing that extends over the second end of the collar and causes the valve to move to the second position when twisted to separate the frangible webbing from the injection device to expose the hypodermic needle.

### REFERENCE TO RELATED APPLICATIONS

This application claims the benefit of my provisional application having Ser. No. 61/638059, filed Apr. 25, 2012, which is incorporated herein by reference in its entirety.

### BACKGROUND OF THE INVENTION

(a) Field of the Invention

This application relates to a pre-filled, single dose, injection device. More particularly, but not by way of limitation, to

(b) Discussion of Known Art

The need to store a spare key for an automobile has been long recognized.

Therefore, a review of known devices reveals that there remains a need for a simple device that supports a concealed hook for hanging ornaments.

### SUMMARY

It has been discovered that the problems left unanswered by known art can be solved by providing tool support for holding tools against the turret of a turret lathe, the tool includes:

Prefilled Medical Injection Device

### FIELD OF THE INVENTION

The present invention generally relates to a device or system for delivering a dose of medication, vaccine or other infusate product (hereinafter “medication”). More particularly, a hermetically sealed ampoule prepackaged with medication that incorporates an insertion technology consisting of a specially designed collar integrating a variety of possible connections, including needle free connectors, such as luer-lok and luer slip, as well as staked needle, and needle with an active or passive safety system.

Additionally the device is optimally suited for production through the use of the Blow-Fill-Seal manufacturing technique.

### BACKGROUND OF THE INVENTION

There are pluralities of the hypodermic syringe type injection device. The vast majority of the devices are derivative of French physician Charles Pravaz' well-known design. The design consists of a cylinder body, a piston, and a hypodermic needle. This design works very well for introducing and extracting fluids from patients, and has been adapted recently as a prefilled delivery method made suitable by following specific predetermined safety protocols that add significant cost to the infusion equation.

Prefilled medical injection devices provide health care workers with a more efficient way to administer medications. The ubiquitous hypodermic syringe has seen a multitude of incremental advancements and improvements over the years in order to deal with the myriad of problems hypodermic syringes present in its manufacture, distribution, storage and use. Many of these advancements are unique to the problems relating to the prefilled syringe, which creates many new challenges because of the prefilled format.

For example, prefilled hypodermic syringes face problems relating to chemical interactions with silicone, a common lubricant that allows the plunger to move down the cylinder, as well as adhesives and tungsten. Further, transportation presents additional problems relating to atmospheric changes, creating a potential for pressure increases inside the cylinder body causing the device to extrude medication, wasting medication, while decreases cause the device to suction up outside air, which increases the risk of contamination. Many other problems exist in their use, such as the accidental removal or dislodgement of the plunger, as well as the potential for needlestick injury depending upon the type of needle used. This has been a problem to many health care professionals and has forced them to switch back to the vial syringe method for delivering vaccinations.

Although, there are multitudes of prior arts that incorporate solutions to the above problem the majority of them still carry a major disadvantage. The majority of injection devices require that the device be uncapped and recapped to avoid sticking others with a contaminated needle. The requirement to recap a syringe has created a major risk factor for healthcare workers, accidentally pricking themselves with a soiled needle.

It is therefore an object of the present invention to introduce a device that is prefilled with medication, adjusts to changes in ambient temperature and pressure, while providing a risk reducing mechanism to inject patients. Additionally the invention is designed for a mass production method (Blow-Fill-Seal method) that minimizes the risk of contamination. Blow-Fill-Seal has numerous requirements and limitations and the ampoule and collar (insertion technology) have both been designed to be compatible with Blow-Fill-Seal manufacturing.

### BRIEF DESCRIPTION OF THE DRAWINGS

FIG. 1 is a perspective view of the invention in its current embodiment.

FIG. 2 is a front elevational view of the invention in its current embodiment

FIG. 3 is a cross sectional view of the invention in its current embodiment.

FIG. 4 is a cross sectional view of the integral components of the invention in its current embodiment.

FIG. 5 is a perspective view of the disassembled integral components that are utilized in the invention in its current embodiment.

FIG. 6 is a cross sectional view of the disassembled integral components that are utilized in the invention in its current embodiment.

FIG. 7 is a cross section illustration of the invention in with the frangible web, integrated needle assembly, and rotating seal activation mechanism, in the closed position, installed in the collar.

FIG. 8 is a cross section illustration of the invention in the without the frangible web integrated needle assembly, and rotating seal activation mechanism, in the open position, installed in the collar.

FIG. 9 is a cross sectional illustration of the invention with frangible webbing and integrated needle safety assembly, needle cap and rubber stopper.

FIG. 10 is a cross sectional illustration of the invention with a frangible webbing and the integrated needle safety assembly without the needle cap and rubber stopper.

### DETAIL DESCRIPTION OF THE INVENTION

The apparatus is to be described in detail and is provided in a manner that establishes a thorough understanding of the present invention. There may be aspects of the present invention that may be practiced without the implementation of some features as they are described. It should be understood that some details have not been described in detail in order to not unnecessarily obscure focus of the invention.

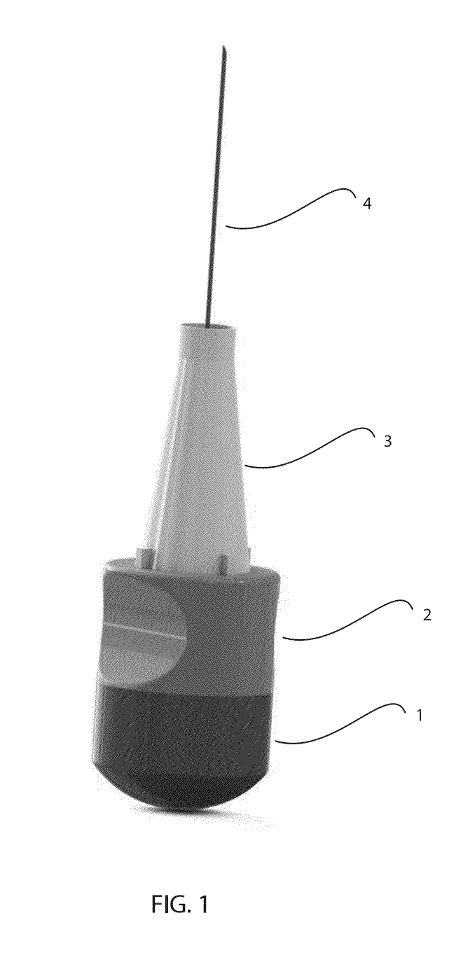

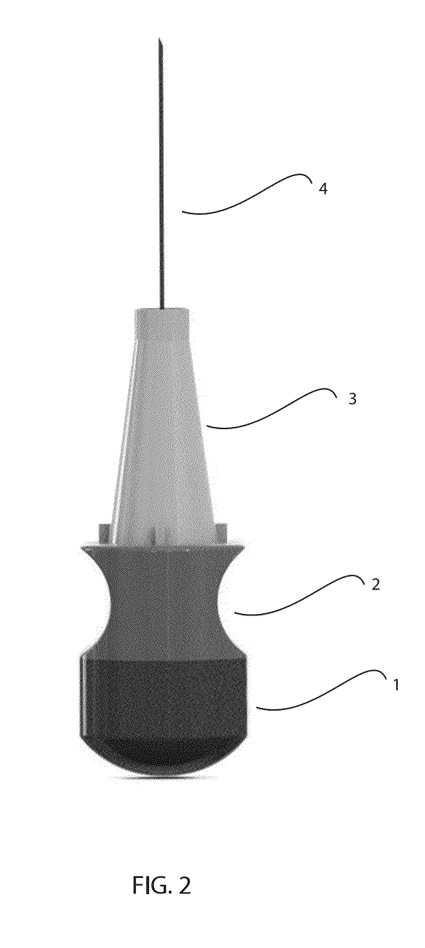

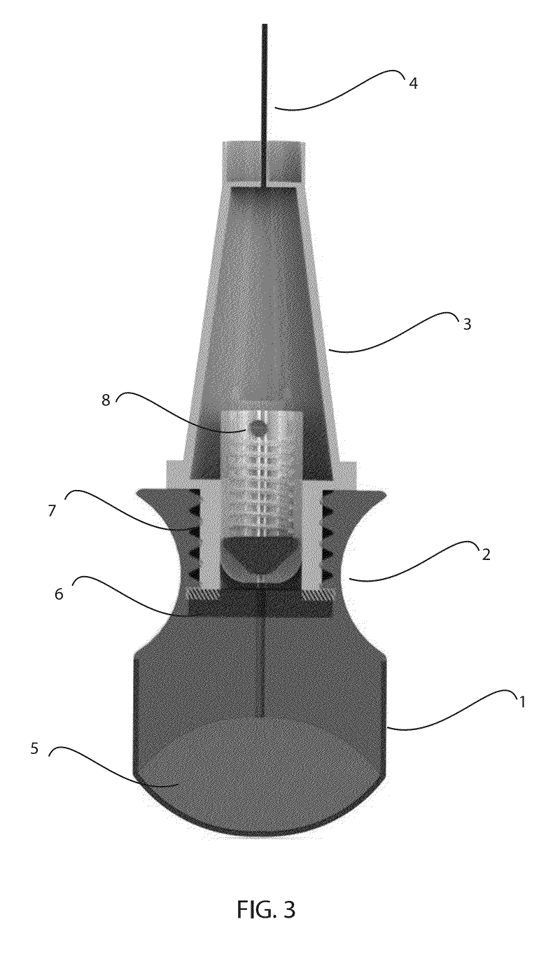

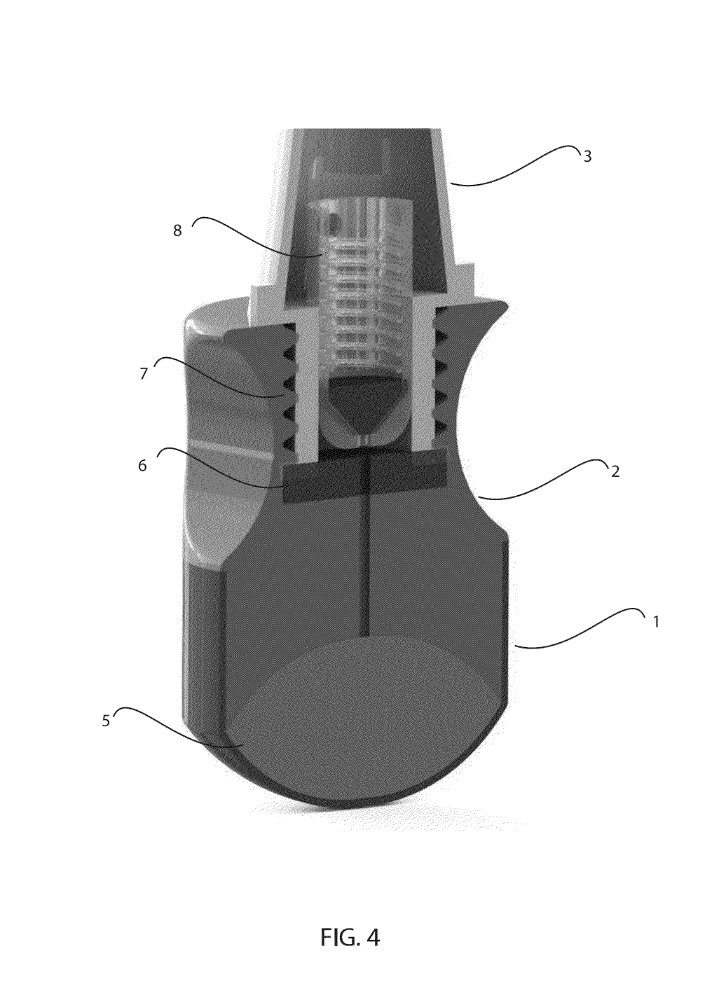

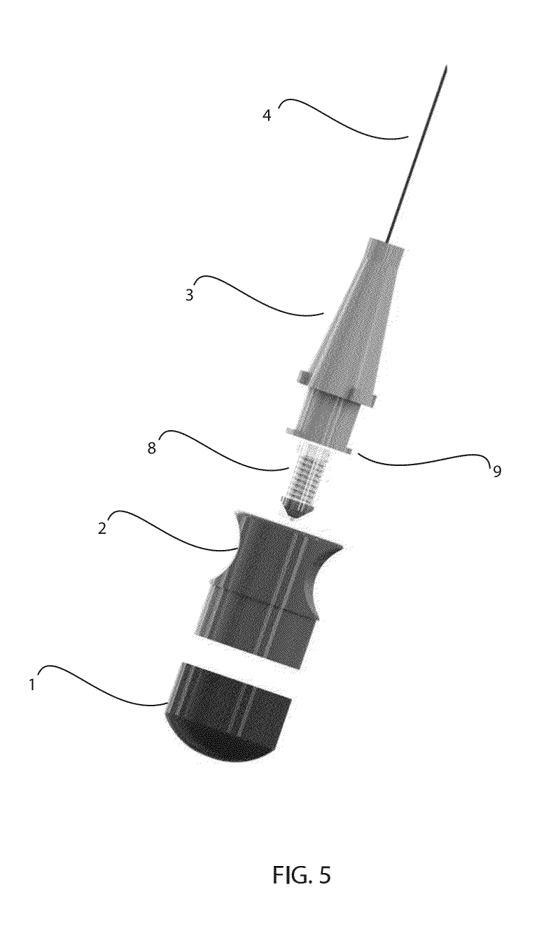

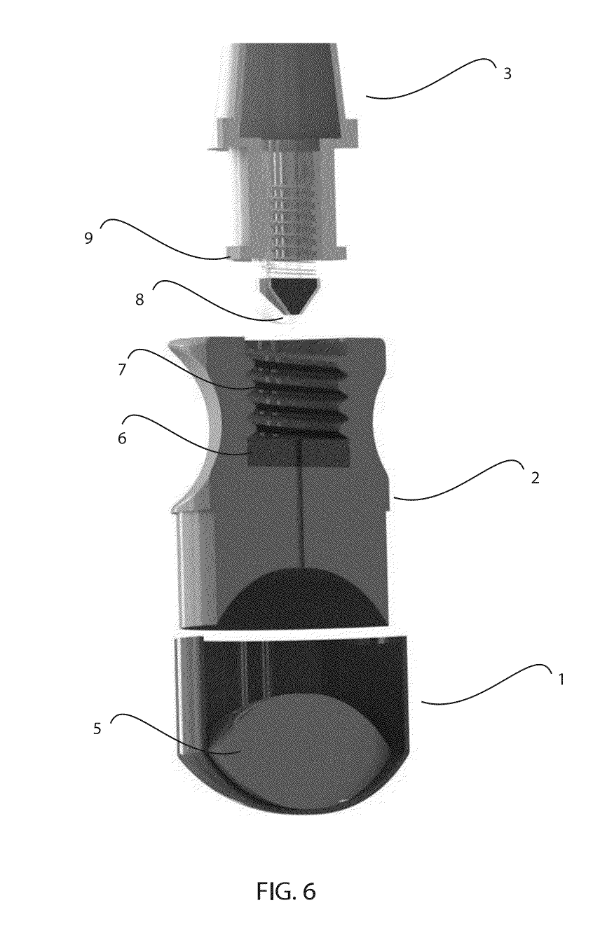

FIG. 1 The present invention includes an ampoule that is prepackaged with medication. The device 100 may include a variety of safety features and is intended to be produced using the Blow-Fill-Seal (BFS) manufacturing process. The device is composed of a hermetically sealed ampoule, 1, with pre-packaged medication, 5, integrated into a collar, 2. The collar, 2, contains a threaded Luer-lok connection (or other connection depending upon the connection type), 7. The device is built with a needle safety system, 3, that re-shields the needle, 4, after use. The safety mechanism, 3, connects to the collar, 2, through the use of the built in Luer-lok threads, which may integrate needle with collar, valve, activation and aspiration components, 7. The device is intended to be used for performing intradermal (ID), intravenous (IV), intramuscular (IM), and subcutaneous (subcut) injections. The device's small size and disposable nature makes it ideally suited for vaccinations.

The device is produced using the Blow-Fill-Seal (BFS) manufacturing process. The component of the device is composed primarily of Low Density Polyethylene (LDPE), although it can be made from any of the wide variety of materials 5 available such a HDPE, Polypropylene (PP), PE, and other suitable materials, 8. The device 100 incorporates the needle, 4, and the medication, 5. The manufacturing process creates the device by extruding the LDPE in a tubular shape. The tubular piece of LDPE is referred to as the parison. The parison is extruded into a mould at which point the mould closes and the LDPE is cut at the top. A vacuum is applied to the exterior of the mould and for larger sizes, may be combined with an internal pressure via a mandrel (nozzle) which blows sterile filtered air. This causes the hot parison to take the shape of the mould. At this point the medication, 5, is delivered into the container through the use of an electronic fill system that utilizes the same mandrel used to optionally blow the air. The electronic fill system delivers a precise dose of the drug into the container through the mandrel. When the precise dose has been transferred, the mandrel retracts from the container. Once the mandrel is removed a premade insert 3, with valve, 8, and collar, 2, (and whatever needle or needle free mechanisms) are correctly oriented into position through the use of a vacuum pick up. Once in place the vacuum pick up retracts and a separate seal mould comes in to hermetically seal the top of the device and create the frangible web, 12. To accomplish this needle safety system, 3, with valve, 8, and collar, 2, insert must be compatible with both diameter and the height of the seal mould. Once all components are sealed the moulds open and the container with the attached assembly is removed. The production process integrates the manufacture of the ampoule, along with the filling and finish operations into a single aseptic machine that requires no human interaction and thereby reduces the risk of contamination.

The ampoule, 1, of the invention, in its present embodiment, is a cup shaped receptacle composed of translucent LDPE with a wall thickness of 0.025-0.012″. The external dimension of the ampoule is 0.5″ in diameter (can be larger). The ampoule can hold medication volumes that range from 0.1-5 mL. When being filled the ampoule, 1, must leave about 0.5 ml of headspace with a 0.5 ml dose. This head space is necessary during the fill however creates a problem at time of injection into patient which is overcome at time of use with an aspiration function so that 0.5 ml of nitrogen can be aspirated. The addition of nitrogen provides better stability for the storage of the drug, as nitrogen is an inert gas. Typical overfill must be between 5% and 10% but maybe more with a medication 5 size of 0.1 mL.

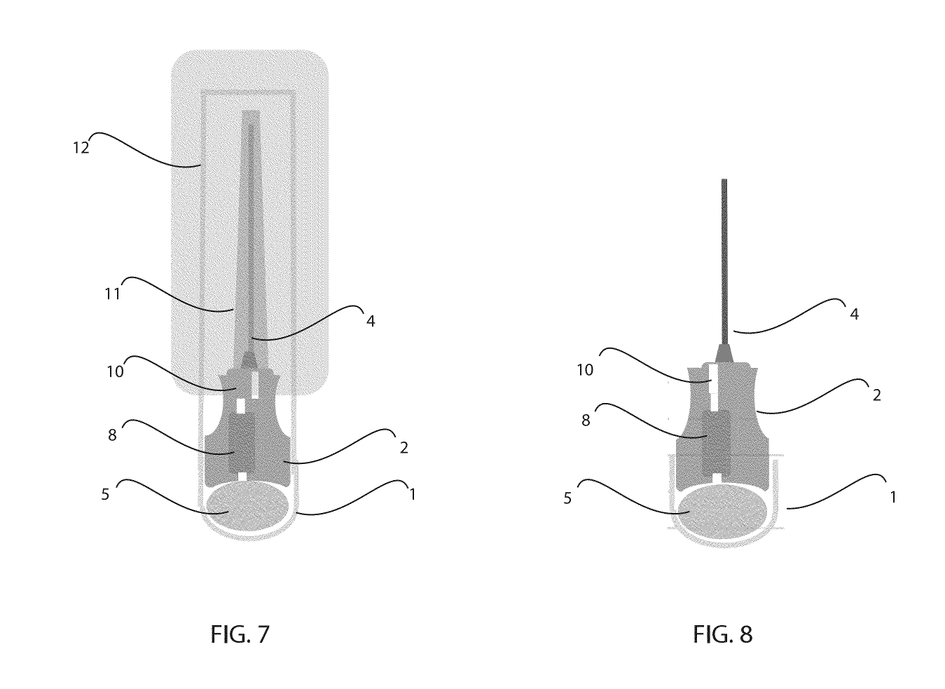

The collar, 2, of the invention in its present embodiment, is a cylinder with two concavities positioned bi-laterally. The bilateral concavities serve as a point of contact for the user's fingers when the invention is held. The collar, 2, can be composed of LDPE, High Density Polyethylene (HDPE), or Polypropylene (PP), and with an intended diameter of ˜0.5″ and 0.75″ in length. The collar, 2, has four configurations that determine the overall positioning of internal components. The differences in the configuration are not exclusive to their individual embodiments and the features can be combined to obtain the optimal utility for the device. The collar, 2, configurations that differ are threaded, integrated, and rotating seal activation mechanism, 10, that can be incorporated with either an integrated, 11, or Luer-lok threaded, 7, needle, 4. The threaded Luer-lok collar, 2, 7, has a cavity distal to the ampoule, 1, attachment. The threaded cavity contains a silicone seal, 6, on the bottom that assists in sealing the end face, 9, of the safety needle, 3, assembly. The threaded Luer-lok collar, 2, 7, allows a user to substitute the safety needle, 3, assembly with other attachments that accept a Luer-lok connection, 7. An example of a possible substitution is a needleless heparin-lok, used for heparin flushes. The integrated needle system collar assembly, 2, 4, 11, contains the needle, 4, with safety mechanism attached permanently, 11. FIG. 7, 8 show the collars, 2, containing a rotating seal activation mechanism, 10, can utilize either the Luer-lok safety needle attachment, 3, 7, or have a safety needle permanently integrated, 4, 11, into the collar, 2. The rotating seal activation mechanism, 10, has to reposition the location of the integral valve, 8, from being inside the safety needle mechanism, 3, to being underneath the rotating seal activation mechanism, 10. The rotating seal activation mechanism, 10, is attached to the frangible webbing, 12. Removal of the frangible webbing, 12, rotates the seal activation mechanism, 10, to align with the integral valve, 8, and the needle, 4, allowing for the extrusion of the medication, 5.

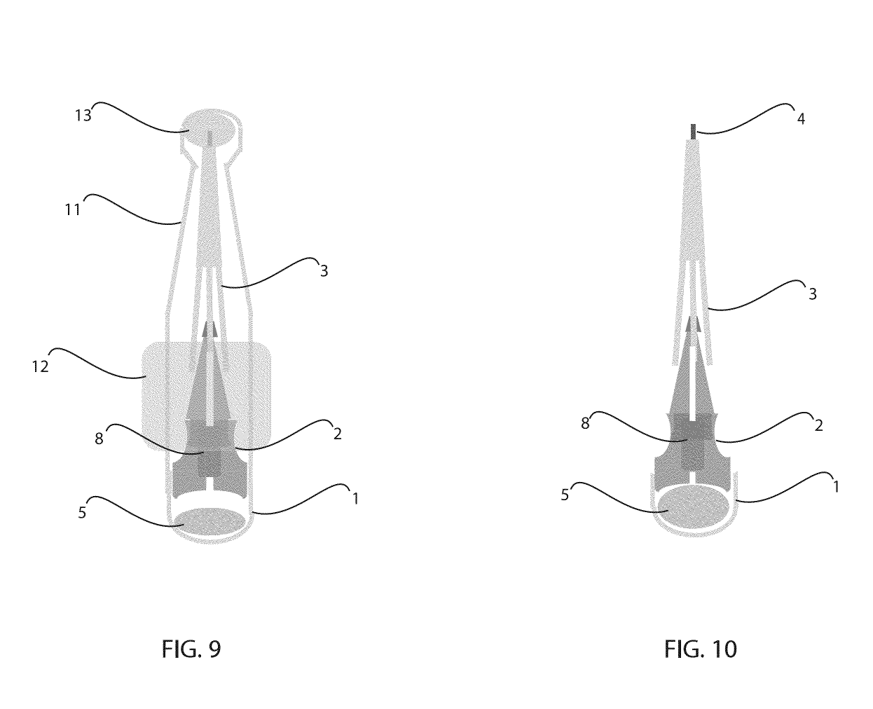

FIG. 9 shows the needle safety mechanism, 3, in the present embodiment of the invention is a Novaguard safety needle. Although, Novaguard is the current needle safety system that is being utilized, the device's incorporation of a Luer-lok threading, 7, allows for a plurality of needle attachments. The Novaguard design keeps the needle housing, 3, the needle, 4, needle cap, 11, and rubber stopper, 13. Additionally 5 when the needle housing, 3, is attached to a collar, 2, without a rotating seal activation mechanism, 10, a polycarbonate integral valve, 8, with 4 psi cracking pressure is placed inside the needle housing, 3, to prevent the medication, 5, from being extruded and to provide cracking pressure to prevent accidental discharge during use. When the safety needle housing assembly, 3, is connected to a collar, 2, and ampoule, 1, a frangible web, 12, is used to seal it.

Although the invention has been explained in relation to its preferred embodiment, it is to be understood that many other possible modifications and variations can be made without departing from the spirit and scope of the invention as herein described.

What is claimed is:

1. An integrated injection device with pre-packaged medication, the integrated injection device comprising:
an ampule that is filled with a fluid medication;
a collar and hypodermic needle, the collar having a first end that is adapted for accepting the ampule, and a second end that includes an exit aperture, the collar further comprising a valve that is movable from a first position where the valve blocks fluid communication between the first end and the second end and a second position where valve provides fluid communication between the first end and the exit aperture in the second end.

2. An integrated injection device according to claim 1 wherein said injection device is encapsulated by a frangible webbing packaging and a hypodermic needle that extends from the second end of the collar, the frangible webbing packaging including an area with a reduced cross section that extends over the first end of the collar, so that twisting of the frangible webbing packaging results in severing a portion of the frangible webbing to expose the hypodermic needle and in moving the valve from the first position to the second position.

3. An integrated injection device according to claim 2 wherein said valve is movable about an axis that extends between the first end and the second end, so that twisting of the frangible webbing about the axis that extends between the first end and the second end to sever the frangible webbing rotates the valve to the second position as the frangible webbing is severed.

4. An integrated injection device according to claim 3 wherein said ampule that holds medication is an independent bladder.
